(19) United States
(12) Patent Application Publication
(10) Pub. No.: US 2019/0385751 A1
(43) Pub. Date: Dec. 19, 2019

(54) DISEASE CONDITION INFORMATION RESEARCH METHOD AND SYSTEM, AND STORAGE MEDIUM

(71) Applicant: Yifan ZHU, Kaifeng (CN)

(72) Inventors: Yifan ZHU, Kaifeng (CN); Yanping CAO, Kaifeng (CN); Xiaoxiao ZHANG, Chengdu (CN)

(21) Appl. No.: 16/119,710

(22) Filed: Aug. 31, 2018

(30) Foreign Application Priority Data
Jun. 15, 2018 (CN) 2018106231845

(51) Int. Cl. G16H 70/60

(52) U.S. Cl. CPC G16H 70/60

(57) ABSTRACT

The present invention provides a disease condition information research method and system. The system includes: an information collection module, configured to collect clinical manifestation information in different forms; an information analysis module, configured to establish logical association among the cause-pathophysiology-clinical manifestation information by using the information collected by the information collection module according to the objective laws of medical knowledge so as to form a disease condition information analysis model; and a result display module, configured to display pathophysiological and anatomical location lesion information after the information analysis module performs disease condition analysis. The present invention can help the medical students to more deeply understand the teaching contents of physiology, anatomy, pathology, pathophysiology, clinical diagnostics, surgery and internal medicine, thus improving the teaching quality.

### FIELD OF THE INVENTION

The present invention relates to a disease condition information research method.

### BACKGROUND OF THE INVENTION

Case discussion and research, especially the discussion and research of intractable cases, are important means for medical students and doctors to master and improve their diagnosis and treatment knowledge and skills, broaden their horizons and increase their experience. From the perspective of medical principles, the case research involves a large amount of basic medical knowledge and clinical medical knowledge. As to the research contents, the causes of anatomy, physiology, pathology, pathophysiology, medical history, symptoms, signs and abnormal examination results are inevitable contents of the case research.

However, due to the long history, the medical students and the doctors have forgotten a lot of basic medical knowledge they studied many years ago; in addition, with the progress of the medical research, a lot of medical foundations and clinical knowledge are constantly updated, clinicians are busy and cannot update their medical foundations and clinical knowledge in time, resulting in knowledge aging and skill lagging, and reducing the clinical diagnosis and treatment efficiency and accuracy.

In order to help the medical students to better understand the diseases and understand the medical knowledge, and to help the doctors to grasp the latest development in the discipline with higher efficiency and understand the diseases from the perspective of the mutual relationship between the medical basic knowledge and the clinical knowledge, according to 30 years of experience and theoretical accumulation in medicine practice and medical research, the inventor provides a series of content of the present invention.

### SUMMARY OF THE INVENTION

The present invention provides a method capable of analyzing and intelligently judging the pathophysiology corresponding to clinical manifestation according to objective inherent laws of medical knowledge.

The present invention adopts the following technical solution:

A disease condition information research method includes:

collecting different clinical manifestation information, and establishing a disease condition information analysis model according to anatomical locations of possible clinical causes corresponding to the clinical manifestation information and according to the logical association among the cause-pathophysiology-clinical manifestation information;

inputting the clinical manifestation information of a patient into the disease condition information analysis model, and associating the clinical manifestation with the cause by using the applicable pathophysiological knowledge according to the logical association of medical knowledge objective laws among the cause-pathophysiology-clinical manifestation information; and

displaying the applicable pathophysiological information in the form of text or sound or image.

When the disease condition information analysis model is applied to disease condition information analysis according to the clinical manifestation information, the anatomical locations of the possible clinical causes corresponding to the clinical manifestations are listed at first, if the applicable pathophysiological knowledge associating possible lesions and the clinical manifestations exists in an existing known pathophysiological knowledge base of human, the clinical manifestations are correctly associated with the possible causes, or otherwise, the clinical manifestations are not correctly associated with the possible causes.

The clinical manifestation information includes medical history information, symptom information, physical sign information and examination and test result information.

A system applying the above method includes:

an information collection module, configured to collect clinical manifestation information in different forms;

an information analysis module, configured to establish logical association among the cause-pathophysiology-clinical manifestation information by using the information collected by the information collection module according to the objective laws of medical knowledge so as to form a disease condition information analysis model; and

a result display module, configured to display pathophysiological and anatomical location lesion information after the information analysis module performs disease condition analysis.

The system further includes an anatomical location lesion knowledge base and pathophysiological knowledge base updating module, configured to input the clinical information collected by the information collection module to the information analysis module and update the data of the disease condition information analysis model in the information analysis module.

A storage medium storing a computer program, wherein the computer program implements the method according to claims 1-3 when being executed by a processor.

The present invention has the following beneficial effects:

(1) The present invention can help the medical students to more deeply understand the teaching contents of physiology, anatomy, pathology, pathophysiology, clinical diagnostics, surgery and internal medicine, thus improving the teaching quality;

(2) the lesion knowledge base of the anatomical locations and the corresponding pathophysiological knowledge base, which are constantly updated, in the present invention can help the doctors to keep abreast of the latest progress of researches in related professional fields and master the development frontier knowledge of the subject; and

(3) a cause can trigger different clinical manifestations according to different pathophysiology. The present invention can help the doctors to fully understand the pathophysiological conditions of the patient, deepen the understanding of the disease, and contribute to the training, cultivation and clinical works of the doctors.

### DETAILED DESCRIPTION OF THE EMBODIMENTS

The present invention will be further described in detail below in conjunction with specific embodiments.

The present invention provides a disease condition information research method, including the following steps:

establishing a disease condition information analysis model according to the medical objective law that different causes implement different pathophysiological functions to produce corresponding clinical manifestations.

Inputting the clinical manifestation information of a patient into the disease condition information analysis model, and associating the clinical manifestation with the cause by using the applicable pathophysiological knowledge according to the logical association of medical knowledge objective laws among the cause-pathophysiology-clinical manifestation information; and

displaying the applicable pathophysiological information in the form of text or sound or image. The applicability herein means meeting the correct association of the medical laws.

In the present invention, since the cause is objectively embodied in a certain anatomical location of the human body, that is, the pathological change of the anatomical location is a specific manifestation of the cause, the data in the disease condition information analysis model is composed of (1) anatomical location, (2) pathophysiology and (3) clinical manifestations.

The logical association among the three types of data is the medical objective law of the intrinsic association of cause-pathophysiology-clinical manifestation, a model is established according to the medical objective law, the clinical manifestation information of the patient is input to the model via the model, the pathophysiology corresponding to the clinical manifestation information and corresponding possible lesion information of the anatomical location are obtained by comparing the lesion knowledge base of the anatomical location and the corresponding pathophysiological knowledge base, and then the pathophysiology is displayed for the research and study by professionals.

The disease condition information analysis model is established according to the existing confirmed cases and the medical objective rule of the above-mentioned cause-pathophysiology-clinical manifestation, after the model is stored in the form of a database, a disease condition information analysis database is formed, the database may include multiple databases, for example, the database may include an anatomical location lesion database, a pathophysiological database and a clinical manifestation database, and the databases has the above-mentioned intrinsic association meeting the medical objective rule. After the data of the patient is input, the clinical manifestations are associated with the anatomical location lesions through appropriate pathophysiological knowledge by calling the knowledge points in the databases.

When the disease condition information analysis model is applied to disease condition information analysis according to the clinical manifestation information, the anatomical locations of the possible clinical causes corresponding to the clinical manifestations are listed at first, if the applicable pathophysiological knowledge associating possible lesions and the clinical manifestations exists in an existing known pathophysiological knowledge base of human, the clinical manifestations are correctly associated with the possible causes, or otherwise, the clinical manifestations are not correctly associated with the possible causes.

The above-mentioned clinical manifestation information at least includes symptom information and physical sign information obtained by query and observation of the doctor, and information obtained according to the examination and test results of instruments.

The present invention further provides a disease condition information research system, including:

an information collection module, configured to collect clinical manifestation information in different forms, such as sound, text, image and the like, and the information is input to the system as sample information;

an information analysis module, configured to establish logical association among the cause-pathophysiology-clinical manifestation information by using the information collected by the information collection module according to the existing medical knowledge objective laws so as to form a disease condition information analysis model; and

a result display module, configured to collect an image, a checklist result and a test list result through the patient's chief complaints, previous history, current medical history, and the sound and text (inputting and scanning) describing symptoms and signs collected by the information collection module, and then inputting the clinical manifestation information of the patient in the disease condition information analysis model of the information analysis module, and displaying pathophysiological information after the disease condition analysis.

Because medical knowledge is always evolving, the system further includes an updating module configured to update the database of the disease condition information analysis model.

The above-mentioned information analysis module is the core of the present invention, the function of the module is specifically reflected in an algorithm for analyzing and researching the disease condition information, and the algorithm is obtained based on the in-depth understanding of the medical knowledge:

(1) when a human body is sick, the cause triggers a series of clinical manifestations such as symptoms, signs and abnormal examination and test results through its pathophysiological function;

(2) the cause of the disease is objectively present in vivo, and is specifically embodied in the pathological changes of a certain anatomical location; and

(3) furthermore, the pathological changes (causes) of the certain anatomical location establish an intrinsic logical association corresponding relationship meeting the objective laws of medical knowledge through the pathophysiology and clinical manifestations thereof.

Therefore, the implementation of the information analysis module is embodied in two small module functions: (1) a disease condition information classification module, and (2) a disease condition information association module.

(1) The disease condition information classification module: according to the above-mentioned in-depth understanding of the medical knowledge, the module lists the chief complaints, symptoms, signs, abnormal examination results and the like in a clinical manifestation column one by one, and lists the anatomical locations that may have lesions corresponding to the clinical manifestations in the column.

(2) the disease condition information association module: according to the above-mentioned in-depth understanding of medical knowledge, and according to the medical pathophysiological knowledge that has been mastered by the human beings at present, if the clinical manifestations are associated with the possible lesions on the anatomical location by appropriate pathophysiological knowledge, the disease condition research is correct. Otherwise, the disease condition research is incorrect.

The present invention interprets all clinical manifestations and pathophysiological manifestations with one lesion or as few lesions as possible when obtaining pathological physiology and the possible lesions of the anatomical location according to clinical manifestation data.

A specific embodiment of the present invention is described below:

Case: Hypophysoma

Male patient, 74 years old. There was no obvious cause of dizziness and vomiting a month ago. When the patient visits a local clinic, the measured blood pressure is 180/110 mmHg, and the patient is subjected to infusion therapy (unknown drug), but the symptoms are not relieved. The patient visited the outpatient clinic of our hospital 3 days ago and was subjected to antihypertensive treatment (irbesartan hydrochlorothiazide tablets), and after the medicine was taken, the blood pressure of the patient increased, and the dizziness was increased, the patient vomited brown matters accompanied by shortness of breath, chest distress, and paralysis of both lower limbs, which are aggravated at night, as well as discomfort of hypochondria on both sides, and for further treatment, the patient was admitted in the digestive department for the diseases of “acute gastritis and hypertension”.

In the past 15 years, the patient suffered vomit and hyponatremia, for multiple times and the causes have not been clarified, and the patient got better after being treated with symptomatic sodium supplementation. The patient has no history of hypertension, diabetes, coronary heart disease, surgery, etc.

Physical examinations: the body temperature is 36.1° C., the pulse is 75 beats/min, the breathing is 20 beats/min, and the blood pressure is 1180/110 mmHg. The physical examinations are normal, and there is no abnormal positive finding.

Acute blood test and renal function are normal, the blood sodium concentration is 119 mmol/L, the potassium concentration is 3.25 mmol/L, and the chlorine concentration is 85 mmol/L. The patient is subjected to blood pressure control, acid suppression, fluid replacement, electrolyte imbalance correction and symptomatic treatment after admission.

Analysis: the symptoms, signs and positive examination results are classified.

TABLE 1
cause association graph

| y′ | | f′ | x′ |
|---|---|---|---|
| Symptom, sign, and test finding | | Pathophysiology | Anatomical location |
| Dizziness | | ? | Brain, balance organs, |
| | | | and heart and cerebral |
| | | | vessels |
| Vomiting | | ? | Stomach and intestine |
| Deterioration after taking medicine | | Side effect | Medicine |
| High blood pressure | | ? | Angiocarpy |
| High blood | suffering Vomit and hyponatremia for | ? | Kidney, stomach and |
| pressure | multiple times in the past 15 years | | intestine |
| | Blood sodium, potassium and chlorine | ? | |
| | are lower than normal values | | |

1. The dizziness and vomiting of the patient involve a variety of tissues and organs, and further examination must be made.

2. The symptoms are aggravated after taking the medicine, which may be caused by the side effects of the medicine.

3. The patient did not complain about the history of hypertension, and the cause of the hypertension must be investigated.

4. In the past 15 years, the patient suffered vomit and hyponatremia for multiple times, and got better after being treated with symptomatic sodium supplementation, which indicates that vomiting is highly correlated with the hyponatremia, and the present vomiting with hyponatremia should be a consistent manifestation of the disease condition of the patient. The maintenance of the blood sodium level depends on intake and discharge. The cause of the hyponatremia may be less intake or more discharge. Careful query of the treatment of the patient during the past 15 years shows that there is no obvious cause of the nausea and vomiting, and the hyponatremia is discovered after the treatment. The vomiting is transient, no long-term vomiting is generated, and the patient's diet, urine and stool are normal. Therefore, it is concluded that the hyponatremia is not caused by the vomiting and under-intake, the hyponatremia caused by gastrointestinal factors is excluded, and the kidney condition should be examined primarily. The sodium sulphate is 413.8 mmol/24 hours (130-127 mmol), it is clear that the sodium removal of the kidney increased, but the renal function and the urine routine are normal, so the factor of the kidney is excluded.

From the physiological point of view of blood sodium metabolism, there is a thalamic-pituitary-adrenal axis. The main physiological function of the adrenal mineralocorticoid is to promote the renal tubular reabsorption of sodium, the adrenal dysfunction may cause a decrease in adrenal cortex hormones, resulting in increased renal sodium excretion; in addition, the hypothalamic and pituitary lesions may also affect the function of the adrenal gland, and the patient has dizziness symptoms, it is necessary to perform brain examination. The head CT suggests possible hypophysoma. The visual field examination shows that the left eye is 20o-30o, with a shadow in the superior temporal visual field, the right eye is 0o-30o, with a shadow in the superior temporal visual field, so the patient is transferred to the neurosurgery and is subjected to surgical treatment after the examination; and the pathological examination shows pituitary adenoma.

Analysis points:

After the signs, symptoms and positive findings are classified, according to the diagnostic principle of “explaining multiple clinical manifestations by one cause as much as possible”, the symptoms, signs, and positive findings are interpreted by using physiological, pathological and physiopathological knowledge, and in conjunction the medical history closely, comprehensive analysis is conducted in a comprehensive way and goes deeper.

The disease condition information analysis system of the present invention can be embodied as a medical device integrating the above three module functions, or a set of medical device connecting a disease condition information collection product, a cloud information analysis server (implementing the function of the information analysis module), and a result feedback product.

The present invention further provides a storage medium, storing a computer program, wherein the computer program implements the above-mentioned method when being executed by a processor.

The present invention can also be embodied in the form of a disease condition analysis algorithm chip.

Described above are only preferred embodiments of the present invention. It should be noted that those skilled in the art can make a number of modifications and improvements without departing from the overall concept of the present invention, and these modifications and improvements should also be encompassed within the protection scope of the present invention.

1. A disease condition information research method, comprising
collecting different clinical manifestation information, and establishing a disease condition information analysis model according to anatomical locations of possible clinical causes corresponding to the clinical manifestation information and according to the logical association among the cause-pathophysiology-clinical manifestation information;
inputting the clinical manifestation information of a patient into the disease condition information analysis model, and associating the clinical manifestation with the cause by using the applicable pathophysiological knowledge according to the logical association of medical knowledge objective laws among the cause-pathophysiology-clinical manifestation information;
and displaying the applicable pathophysiological information in the form of text or sound or image.

2. The disease condition information research method according to claim 1, wherein when the disease condition information analysis model is applied to disease condition information analysis according to the clinical manifestation information, the anatomical locations of the possible clinical causes corresponding to the clinical manifestations are listed at first, if the applicable pathophysiological knowledge associating possible lesions and the clinical manifestations exists in an existing known pathophysiological knowledge base of human, the clinical manifestations are correctly associated with the possible causes, or otherwise, the clinical manifestations are not correctly associated with the possible causes.

3. The disease condition information research method according to claim 1, wherein the clinical manifestation information comprises medical history information, symptom information, physical sign information and examination and test result information.

4. A system applying the method according to claim 1, comprising:
an information collection module, configured to collect clinical manifestation information in different forms;
an information analysis module, configured to establish logical association among the cause-pathophysiology-clinical manifestation information by using the information collected by the information collection module according to the objective laws of medical knowledge so as to form a disease condition information analysis model; and
a result display module, configured to display pathophysiological and anatomical location lesion information after the information analysis module performs disease condition analysis.

5. The system according to claim 4, wherein:
the system further comprises an anatomical location lesion knowledge base and pathophysiological knowledge base updating module, configured to input the clinical information collected by the information collection module to the information analysis module and update the data of the disease condition information analysis model in the information analysis module.

6. A storage medium storing a computer program, wherein the computer program implements the method according to claim 1 when being executed by a processor.
